(19) United States
(12) Patent Application Publication
(10) Pub. No.: US 2004/0015049 A1
(43) Pub. Date: Jan. 22, 2004

(54) Endoscope with sideview optics

(76) Inventor: Kersten Zaar, Albstadt (DE)

Correspondence Address: ORUM & ROTH, 53 W. JACKSON BLVD, CHICAGO, IL, 60604, US

(21) Appl. No.: 10/358,598

(22) Filed: Feb. 5, 2003

(30) Foreign Application Priority Data
Feb. 5, 2002 (DE) 102 04 718.9

(51) Int. Cl. A61B 1/00

(52) U.S. Cl. 600/101

(57) ABSTRACT

An endoscope (1) is proposed with a rotatable side-viewing optics system (11), which has a mirror (15) for deflecting light onto an object to be examined and for reflecting the image of the object. A video camera (7) is located inside a hollow cylinder (6) that can be moved in rotation and the side-viewing optics system (11) is mechanically connected to the hollow cylinder (6) on the side that faces away from the viewing direction (12) of the video camera (7).

### STATE OF THE ART

The invention starts from an endoscope with sideview optics according to the category of the main claim.

Endoscopes with sideview systems for viewing and critically examining tubular inside walls and joints are known from the state of the art, with which a desired deviation of the field of view can be achieved. For this purpose optical systems can be used which consist of a deviation optics that can be rigidly mounted to an endoscope or a tubular camera, but which do not have a motor driven swivel head, so that only a small area of the tube inside wall can be examined. To enlarge the field of view of the tubular camera, an optics was created with a continuous 360° panoramic view, which allows a view of the tube inside wall along with the view of the front. Such an enlargement of the field of view is however, accompanied by a very burdensome reduction of the illumination of the wall areas to be examined.

To focus the existing light quantity on smaller areas for the purpose of improving the illumination and still be able to optically examine larger inside wall areas, endoscopes were proposed in which the camera is arranged on a motor driven swiveling and/or tilting device. Such a swiveling device for an endoscope is known from DE-OS 43 29 162, which consists of a disc-shaped end piece connected with three piezoelectric drive elements at three points, on which a camera chip is attached. Depending on the drive of the driving elements, the end piece can be deviated at an angle which is very much smaller than 90°.

This problem of being able to deviate the direction of view by 90° opposite the longitudinal axis of the endoscope is solved by a known endoscope with a tube camera by arranging in the direction of the object to be photographed in front of the camera a motor driven and rotatable mirror around the longitudinal axis of the endoscope which can be swiveled opposite the longitudinal axis in such a way that it can occupy any desired angle opposite it. The motor forms a constructive unit with the mirror, which is connected with the tube camera by means of several crosspieces. In particular with smaller endoscopes these crosspieces may not exceed a certain strength, so that as a result the reflected image of the object to be examined can be disturbed, by for example having deciding damaged areas of the tube inside wall covered by a crosspiece. Moreover, these crosspieces can get into the cone of the light illuminating the inside wall, so that shadows are formed, which can also result in the damaged areas of the object to be examined remaining undetected. Further, there is the danger that crosspieces will reflect the light serving for illumination, so that a video camera with automatic exposure setting picks up the strongly illuminated crosspiece, but not the darker background, i.e. the object to be examined.

### THE INVENTION AND ITS ADVANTAGES

The endoscope of the invention with sideview optics has the advantage over the previously mentioned endoscopes of making possible an endoscopic view of objects that are difficult to access, in which the light serving to illuminate the object reaches the object unhindered and the image of the object to be examined is reflected by a sideview optics to the lens of a video camera used for this purpose undisturbed.

In accordance with an advantageous embodiment of the invention, the video camera is surrounded by a cylinder-shaped casing, which forms a clearance with the hollow cylinder and that on the casing at least one gasket is arranged for sealing of the clearance. This prevents dampness or dirt from penetrating into the interior of the endoscope, which can impair its operability.

In accordance with a further advantageous embodiment of the invention, the electric motor has a gear with which the hollow cylinder can be rotated by means of a hollow gear arranged on its cylinder inner wall. As a result, it is possible to arrange either only the camera or in addition also the electric motor within the hollow cylinder.

In accordance with a further advantageous embodiment of the invention, a transmission is arranged between the electric motor and the gear, with which the speed of the electric motor can be reduced in suitable fashion.

In accordance with a further advantageous embodiment of the invention, the video camera is mechanically connected with the electric motor. This makes it possible to support the rotating hollow cylinder, depending on the embodiment of the invention, either only on the casing of the video camera or on the joint casing of the electric motor and the video camera, which gives the endoscope of the invention a great mechanical strength.

In accordance with a further advantageous embodiment of the invention, the sideview optics is connected with the hollow cylinder by means of a semi-tubular attachment, which permits an unhindered illumination and view of the object to be examined and results in a stable mounting support of the sideview optics on the hollow cylinder.

In accordance with a further advantageous embodiment of the invention, the mirror of the sideview optics is constructed as a prism, which has an improved reflectivity compared with conventional mirrors.

In accordance with a further advantageous embodiment of the invention, the electric motor can propel the hollow cylinder by means of a friction gear, which in particular with small endoscopes can be implemented at a very favorable price.

In accordance with a further advantageous embodiment of the invention, the electric motor is constructed as a stepping motor, which consists of electrical windings arranged on the casing and of permanent magnets arranged on the hollow cylinder that enter into electromagnetic interaction with the windings depending on their drive. Such motors are especially easy to construct and thus can also be manufactured cost-effectively. In particular, however, these motors allow a very exact positioning of the sideview optics of the invention.

In accordance with a further advantageous embodiment of the invention, a device for the coupling of light to the fiber optic light guide with a mounting support of the end of the fiber optic light guide turned toward the photo camera that swivels in such a way that in a first swivel position of the mounting support a maximum and in a second swivel position of the mounting support a minimum light quantity can be coupled in the frontal area of the end of the fiber optic light guide attached to the mounting support. As a result, the quantity of the light coupled in the fiber optic light guide and serving for the illumination of the object to be examined can be precisely adapted to the structure of this object.

In accordance with a further advantageous embodiment of the invention, the device has a lamp with a light-focusing reflector, that the fiber optic light guide is attached to the mounting support with the frontal area pointing in the direction of the reflector, and that the mounting support can be swiveled around a vertical axis lying between the light focus and the reflector between the first and the second swivel positions. As a result of this, it is easy, depending on the swivel position, to have the frontal area of the fiber optic light guide reach the light focus of the reflector in such a way that a precisely measurable light quantity is coupled in the fiber optic light guide.

In accordance with a further advantageous embodiment of the invention, a motor driven rotating arm is provided for swiveling the mounting support, which is mechanically connected with the mounting support by means of a rod and a bolt arranged outside of the axis. Although the of the invention is very simply constructed, it allows a very exact positioning of the frontal area of the fiber optic light guide opposite the cone of light radiated by the reflector.

In accordance with a further advantageous embodiment of the invention, the electric motor is connected with the hollow cylinder by means of a flexible shaft and a miniature gear. This allows the arrangement of the electric motor rotating the hollow cylinder and with it the sideview optics at the distance of the endoscope to any place of the probe, making it possible to further reduce the exterior dimensions of the endoscope. The separation of the mechanically fixed connection between the electric motor and video camera caused by the construction of the invention also makes it possible to move the endoscope through tube turns with a very small radius, to examine their inside area.

In accordance with a further advantageous embodiment of the invention, the camera has an adjustable lens, which, in order to be adjusted, is connected by means of a flexible shaft to an electric motor. This makes it possible to control the adjustment of the lens from the outside, which ensures that the images of the objects to be investigated are always sharp and well recognizable.

Further advantages and advantageous embodiments of the invention can be gathered from the following example description, the drawings and the claims.

### DRAWING

An embodiment of the invention is shown in the drawings and described in greater detail. The drawing show the following:

FIG. 1 an endoscope with a sideview in section.

FIG. 2 a section through a device for coupling of light to a fiber optic light guide along the broken line II-II in FIG. 3,

FIG. 3 top view of the device for coupling of light to the fiber optic light guide and

FIG. 4 an embodiment of the endoscope in which the lens of the video camera and the sideview optics are driven by means of flexible shafts.

### DESCRIPTION OF THE EMBODIMENT

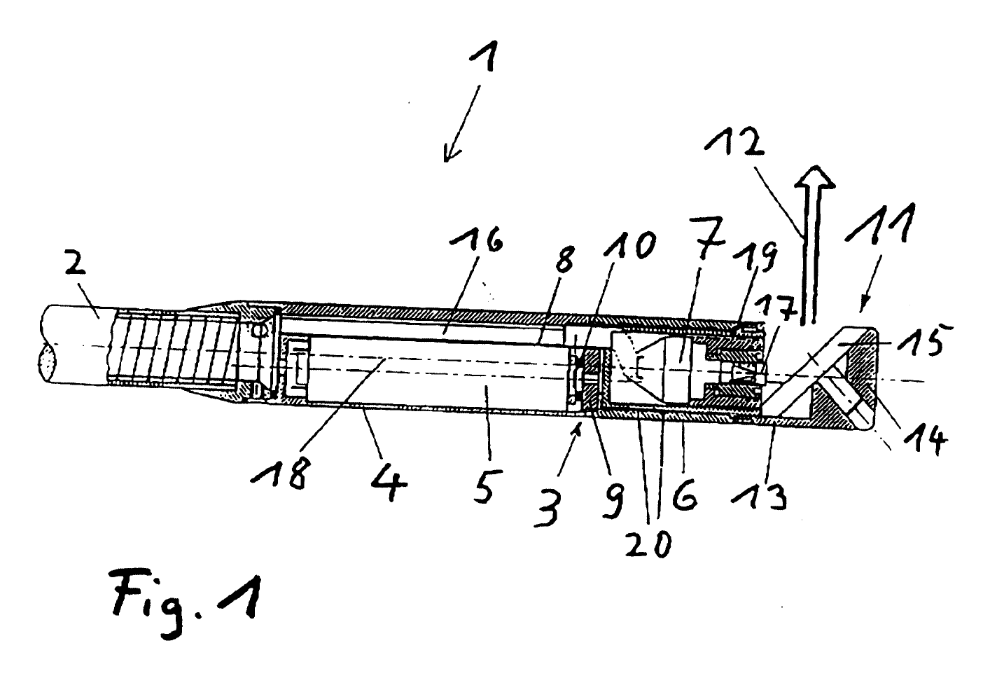

In FIG. 1 an endoscope 1 is shown with a probe 2 and a photo camera 3 attached to it. The photo camera 3 has a casing 4 connected with the probe 2 for an electric motor 5 and a rotating hollow cylinder 6 connected to it, in whose interior a video camera 7 in miniature format is arranged, as normally used for endoscopes and which is not explained in detail here. The video camera 7 is connected to a device for evaluating the supplied video signals by means of an electrical line conducted in the probe 2 and not shown in the figure and is connected with the casing 8 of the electric motor 5, which by means of a gear 9 thus can only rotate the hollow cylinder 6, which for this purpose in the area of the gear 9 has a hollow gear not shown in detail. Further, for this purpose there is a transmission 10 between the electric motor 5 and the gear 9 which reduces the speed of the electric motor 5 in suitable fashion. During one rotation of the hollow cylinder 6 the video camera 7 remains motionless at rest relative to the electric motor 5. In the process the rotary movement of the electric motor 5 and with it of the hollow cylinder 6 can be controlled as desired by means of the conductors guided in the probe 2 which are not shown in the figure. In the present embodiment the video camera 7 is surrounded by a cylinder-shaped casing 19, which forms a clearance with the hollow cylinder. To prevent dampness or dirt from penetrating into this clearance, which could impair the operability of the electric drive (5, 9, 10), gaskets 20 are arranged between the casing 19 and the hollow cylinder 6.

On the side turned toward the direction of view indicated by the arrow 12 a sideview optics 11 is attached to the hollow cylinder 6, which thus turns along with the hollow cylinder 6. The sideview optics 11 consists essentially of a semi-tubular attachment whose one side is connected with the hollow cylinder 6 and on whose other end a mount 14 for a mirror 15 is located, which in the present embodiment is constructed as a prism. With the longitudinal axis 18 of the endoscope 1 the mirror 15 encloses an angle of 45° in the present embodiment. Depending on the purpose of the endoscope 1, however, this angle can also have a different value. If light is added by means of a fiber optic light guide 16 of the video camera 7 conducted laterally on the electric motor 5 and radiated over the frontal area of the fiber optic light guide 16 in a manner not shown in detail, as a result of this the light is deviated and projected in the direction of the arrow 12 on the object to be examined in greater detail. The image of the object illuminated thereby is reflected by the mirror 15 to the lens 17 of the video camera 7 and can thus be picked up by it. In particular when in this fashion the inside wall of a tube is optically scanned, the entire inside wall of the tube to be appraised can be examined by means of a rotation of the sideview optics 11 caused by the electric motor via the hollow cylinder 6.

The electric motor 5 can not only be arranged behind the video camera 7, as shown in the figure, but rather also can be arranged next to it. In this case, which is not shown in the drawing, the video camera 7 is arranged in the same casing as the electric motor 5, and video camera 7 and electric motor 5 are arranged within the hollow cylinder 6 driven by gear or friction wheel by electric motor 5, with the sideview optics being attached to said hollow cylinder.

It is also conceivable to replace the electric motor 5 by means of a stepping motor, in which electrical windings are arranged on the casing 19 of the video camera 7 and the hollow cylinder 6 is equipped with permanent magnets. Depending on the desired direction of view the corresponding electrical windings are triggered, which then exercise attractive or repulsive forces on the permanent magnets and thus turn the sideview optics 11 in the desired position.

The endoscope 1 with the sideview optics 11 of the invention has hereby in particular the advantage that through its attachment to the side turned toward the direction of view by means of the semi-tubular attachment 13 no disturbing attachment crosspieces disturb the path of the rays and the image of the object to be examined.

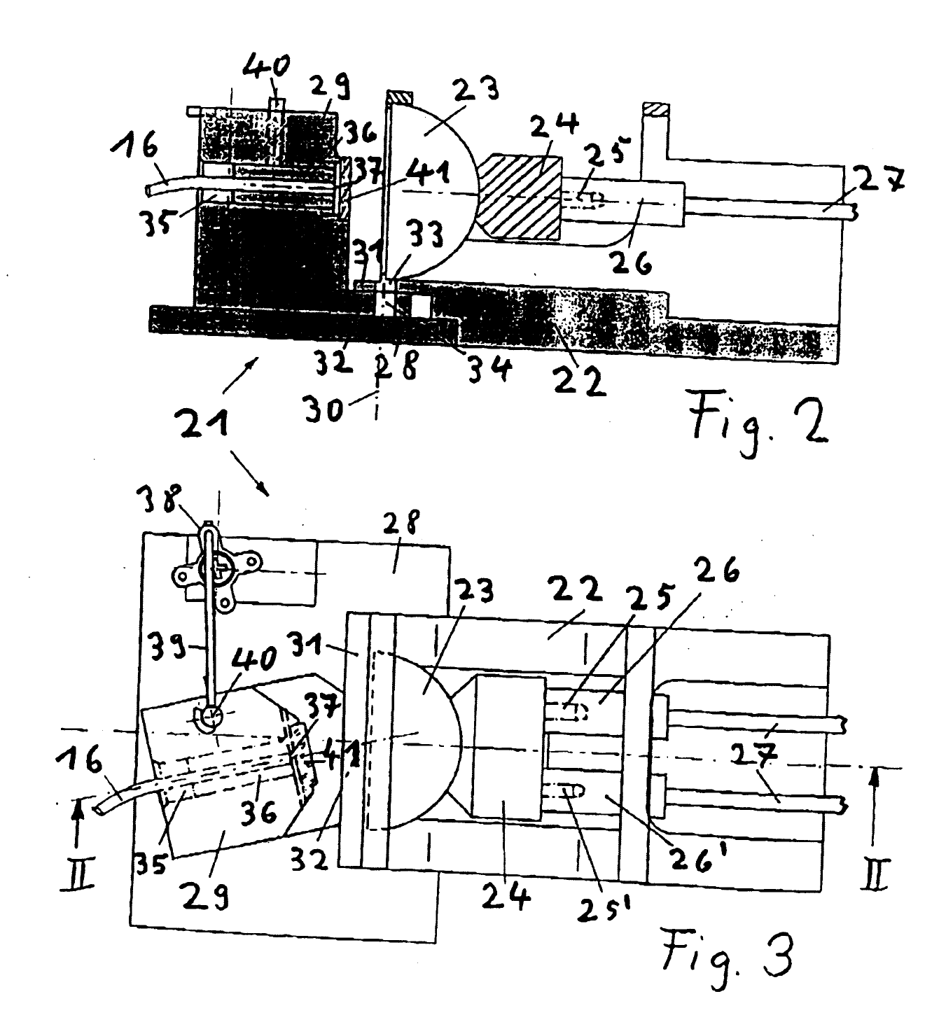

In FIGS. 2 and 3 a device is shown with whose help the light can be coupled in the fiber optic light guide 16, which illuminates the object to be examined with the endoscope 1. In FIG. 3 the device 21 is shown in top view, and FIG. 2 shows a section through the device 21 along the broken line II-II drawing in FIG. 3. The device 21 consists of a lamp attached in a rack 22, of which for simplicity's sake only the reflector 23 is drawn in FIGS. 2 and 3. Said reflector is attached to a lamp bracket 24, which is constructed as an electric plug, whose connecting pins 25 and 25′ each plug into a jack 26, 26′ with connection to the electric power supply 27.

On the side turned toward the power supply 27 a plate 28 is attached to the rack 22, to which a mounting support 29 for the end of the fiber optic light guide 16 is pivoted around a vertical axis 30. A projection 31 of the rack 22 forms with the right end of the plate 28 a recess, in which a projection 32 of the mounting support 29 and by means of a bolt not shown in the figures inserted in the bore 33 of the rack 22 and the bore 34 of the projection 32 is held in such a way with a longitudinal axis lying parallel to axis 30 that rotating motions of the mounting support 29 around the axis 30 are possible.

A bore 35 with a greater diameter is incorporated in the mounting support 29 for the fixation of the end of the fiber optic light guide 16, in which said bore a cylinder-shaped carrier bracket 36 fits, in which the end of the fiber optic light guide 16 is arranged in such a way that its frontal area 37 closes flush with the front side of the carrier bracket 36. Opposite the lamp 23 the frontal area 37 of the fiber optic light guide 16 is arranged in such a way that depending on the swivel position of the mounting support 29 more or less light of the lamp 23 can be coupled in the fiber optic light guide 16. A glass plate 41 is arranged in front of the frontal area 37 for its protection.

The rotating motion of the mounting support 29 is caused by a drive, which has a rotating arm 38 which is mechanically connected with the mounting support (29) by means of a rod (39) and a bolt 40.

In the swivel position of the mounting support 29 shown in FIG. 3, the frontal area 37 of the fiber optic light guide 16 opposite the lamp 23 has a position in which a maximum light quantity can be coupled in the fiber optic light guide 16. With a counter-clockwise rotation of the rotating arm 38 the mounting support 29 is also turned counter-clockwise. This causes the frontal area 37 of the optic light guide 16 to move out from the light focus formed by the reflector 23, thus causing the quantity of the light coupled in the fiber optic light guide 16 to decrease. Thus by adjusting the rotating arm 38 it is possible to precisely set the quantity of light coupled in the fiber optic light guide 16 and adapt exactly to the type of object to be examined by the endoscope 1.

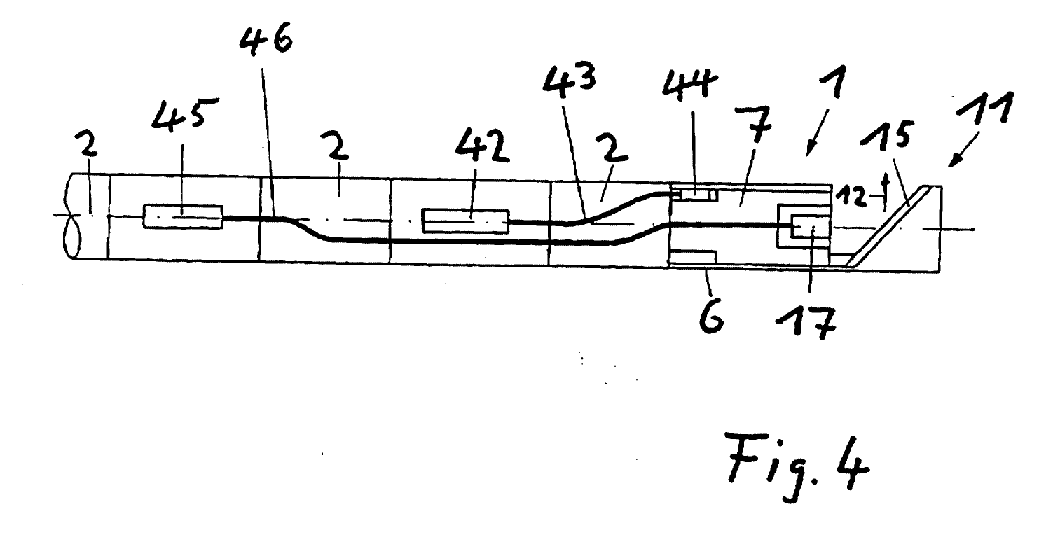

In FIG. 4 a further embodiment of the invention's endoscope with sideview optics 11 is shown. As with the embodiment in accordance with FIG. 1, the video camera 7 with its lens 17 is motionless connected with the probe 2 and arranged within the pivoted hollow cylinder 6, which is connected on the side turned toward the direction of view with the mirror 15 bearing sideview optics 11. In the embodiment according to FIG. 4, however, the hollow cylinder 6 is rotated by an electric motor 42 arranged at a distance to the video camera 7 in the probe 2, whereby the electric motor 42 drives a miniature gear 44 by means of a flexible shaft 43, which then causes the rotation of the hollow cylinder 6. The electric motor 42 can also be arranged outside the probe 2 near to the probe entry.

In the same way the lens 17 is driven by an electric motor 45 by means of a flexible shaft 46 for the purpose of focusing. The power supply and the control of the electric motors 42 and 45 happens in known manner using electric lines and is not shown in FIG. 4. There is also no detailed representation of the flexible shafts 43 and 46 known from the state of the art, which similar to a tachometer shaft from a plastic hose can exist with a pivoted drive spiral within. Finally, it should be pointed out that the length of the probe sections 2 between the video camera 7 and the electric motor 42 and between the electric motors 42 and 45 is variable and can be adapted to the corresponding purpose of the endoscope. Hereby the electric motor 45 can also be arranged outside of the probe 2 near the probe entry. Moreover, it can be advantageous for specific purposes to replace the electric motors 5, 42 and/or 45 with motors that are driven by means of media placed under pressure (e.g. compressed-air motors).

All features portrayed in the description, in the following claims and in the drawings can be essential to the invention both individually and in any given form with each other.

### LIST OF REFERENCE NUMBERS

1 Endoscope

2 Probe

3 Photo camera

4 Casing

5 Electric motor

6 Hollow cylinder

7 Video camera

8 Casing of the electric motor

9 Gear

10 Transmission

11 Sideview optics

12 Arrow, direction of view

13 Attachment

14 Mount

15 Mirror

16 Fiber optic light guide

17 Lens

18 Longitudinal axis

19 Casing of the video camera

20 Gasket

21 Device for coupling of light in 16

22 Rack

23 Lamp, reflector

24 Lamp bracket

25′ Connecting pin

26′ Jack

27 Electric power supply

28 Plate

29 Mounting support

30 Axis

31 Projection of 22

32 Projection of 29

33 Bore

34 Bore

35 Bore

36 Carrier bracket

37 Frontal area of 16

38 Rotating arm

39 Rod

40 Bolt

41 Glass plate

42 Electric motor for the sideview optics 11

43 Flexible shaft for the sideview optics 11

44 Miniature gear for propelling the sideview optics 11

45 Electric motor for setting the lens 17

46 Flexible shaft for the lens 17

1. Endoscope (1) with sideview optics (11),
which is rotated by an electric motor (5, 42), and
which has a mirror (15) for deviation and projection of light in a predetermined direction of view (12) on an object to be examined and for reflecting of the image of the object to a lens (17) of a video camera (7) arranged in an endoscope (1),
characterized by the fact
that the video camera (7) is located in the interior of a pivoted hollow cylinder (6) that can be rotated by an electric motor (5, 42),
that the sideview optics (11) is mechanically connected with the hollow cylinder (6) on the side turned toward the direction of view, and
that the electric motor (5, 42) is arranged behind or next to the video camera (7) in the direction of the object to be photographed by the video camera (7).

2. Endoscope according to claim 1, characterized by the fact that the video camera (7) is surrounded by a cylinder-shaped casing (19), which forms a clearance with the hollow cylinder (6) and that on the casing (19) at least one gasket (20) is arranged for sealing of the clearance.

3. Endoscope according to one of the preceding claims, characterized by the fact that the electric motor (5) has a gear (9) with which the hollow cylinder (6) can be rotated by means of a hollow gear arranged on its cylinder inner wall.

4. Endoscope according to claim 3, characterized by the fact that a transmission (10) is arranged between the electric motor (5) and the gear (9).

5. Endoscope according to one of the preceding claims, characterized by the fact that the video camera (7) is mechanically connected with the electric motor (5).

6. Endoscope according to one of the preceding claims, characterized by the fact that the sideview optics (11) is connected with the hollow cylinder (6) by means of a semi-tubular attachment (13).

7. Endoscope according to one of the preceding claims, characterized by the fact that the mirror (15) of the sideview optics (11) is constructed as a prism.

8. Endoscope according to claim 1 or 2, characterized by the fact that the electric motor (5) propels the hollow cylinder (6) by means of a friction gear.

9. Endoscope according to one of the preceding claims, characterized by the fact that the electric motor (5) is constructed as a stepping motor, which consists of electrical windings arranged on the casing (19) and of permanent magnets arranged on the hollow cylinder (6) that enter into electromagnetic interaction with the windings depending on their drive.

10. Endoscope according to one of the preceding claims, characterized by a device (21) for the coupling of light to the fiber optic light guide (16) with a mounting support (29) of the end of the fiber optic light guide (16) turned toward the photo camera (3) that swivels in such a way that in a first swivel position of the mounting support (29) a maximum and in a second swivel position of the mounting support (29) a minimum light quantity can be coupled in the frontal area (37) of the end of the fiber optic light guide (16) attached to the mounting support (29).

11. Endoscope according to claim 10, characterized by the fact that the device (21) has a lamp with a light-focusing reflector(23), that the fiber optic light guide (16) is attached to the mounting support (29) with the frontal area (37) pointing in the direction of the reflector (23), and that the mounting support (29) can be swiveled around a vertical axis (30) vertical axis (30) lying between the light focus and the reflector (23) between the first and the second swivel positions.

12. Endoscope according to claim 11 or 12, characterized by the fact that a motor driven rotating arm (38) is provided for swiveling the mounting support (29), which is mechanically connected with the mounting support (29) by means of a rod (39) and a bolt arranged outside of the axis (30).

13. Endoscope according to claim 1, characterized by the fact that the electric motor (42) is connected with the hollow cylinder (6) by means of a flexible shaft (43) and a miniature gear (44).

14. Endoscope according to claim 1 or 13, characterized by the fact that the camera (7) has an adjustable lens (17), which, in order to be adjusted, is connected by means of a flexible shaft to an electric motor (45).
